(19) United States
(12) Patent Application Publication
(10) Pub. No.: US 2003/0044389 A1
(43) Pub. Date: Mar. 6, 2003

(54) Microarrays for cell phenotyping and manipulation

(76) Inventors: Patrick O. Brown, Stanford, CA (US); Yoav Soen, Palo Alto, CA (US); Erica Keen, Melrose Park, PA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/190,425

(22) Filed: Jul. 2, 2002

Related U.S. Application Data
(60) Provisional application No. 60/303,109, filed on Jul. 2, 2001.

(51) Int. Cl. G01N 33/567; A61K 45/00

(52) U.S. Cl. 424/93.7; 435/7.21

(57) ABSTRACT

Cells are profiled with respect to their expression of cell surface molecules, and ability to respond to external stimulus in the microenvironment. External stimuli include cell-cell interactions, response to factors, and the like. The cells are arrayed on a planar or three-dimensional substrate through binding to immobilized or partially diffused probes. Probes of interest include specific binding partners for cell surface molecules, signaling cues that act to regulate cell responses, differentiation factors, etc., which may be arrayed as one or a combination of molecules.

### GOVERNMENT SUPPORT

This invention was made with Government support under contract HG009803 awarded by the National Institutes of Health. The Government has certain rights in this invention.

### INTRODUCTION

Living cells utilize unique and elaborate sets of surface molecules, which provide for signaling pathways, interactions with other cells, structural variation, function, and the like. Cell surface receptors allow cells to probe, and to exchange messages with their cellular and extracellular microenvironment. The behavior and fate of a cell is strongly dependent both on the internal state, and on complex cell-cell and cell-ECM interactions mediated by such cell surface molecules. Signal transduction, and the molecules associated with it, comprise a kind of biochemical language. In any multicellular organism, signal transduction messages coordinate activities such as tissue growth, stasis, death and repair.

The ability to screen compounds for biological activity is a multi-billion dollar industry. Many pharmaceutical companies are focused on the identification and validation of therapeutic targets, as well as the identification and optimization of lead compounds. In addition to therapeutics, there is also interest in screening compounds that direct the differentiation and development of cells, such as progenitor cells and stem cells. Factors that act to direct the function of cells, such as chemokines, growth factors, molecules involved in cell death, leukocyte trafficking, and the like, are also of great interest.

The explosion in numbers of potential new targets and chemical entities resulting from genomics and combinatorial chemistry approaches over the past few years has placed enormous pressure on screening programs. The rewards for identification of a useful drug are enormous, but the percentage of hits from any screening problem are generally very low. Desirable compound screening methods solve this problem by both allowing for a high throughput so that many individual compounds can be tested; and by providing biologically relevant information so that there is a good correlation between the information generated by the screening assay and the pharmaceutical effectiveness of the compound.

In addition, cellular physiology involves multiple pathways, where pathways split and join, redundancies in performing specific actions and responding to a change in one pathway by modifying the activity of a different pathway. In order to understand how a candidate drug is acting and whether it will have the desired effect, it is necessary to know, not only the target protein with which the drug reacts, but whether the inhibition of the protein activity will result in the desired response. The development of screening assays that can provide better, faster and more efficient prediction of mechanisms of action, cellular effects and clinical performance is of great interest in a number of fields, and is addressed in the present invention.

Therefore, the ability to (i) identify the type and state of cells, (ii) control their cellular and extracellular microenvironment, (iii) reproducibly manipulate their fate/behavior, and (iv) analyze their interactions with elaborate sets of controlled environments, is of major importance for diagnostic, therapeutic, and research purposes.

### RELATED PUBLICATIONS

A protein microarray is described in International Patent Application WOOO/63701. U.S. Pat. No. 4,591,570 discloses a matrix of antibody coated spots for determination of antigens. Immunophenotyping of cells using an antibody microarray is discussed in Belov et al. (2001) Cancer Research 61:4483-4489. Microarrays of cells expressing defined cDNAs are discussed in Ziauddin et al. (2001) Nature 411:107-110.

### SUMMARY OF THE INVENTION

Compositions and methods are provided for cell profiling, in which cells are profiled with respect to their expression of cell surface molecules, and ability to respond to external stimulus in the microenvironment. External stimuli include cell-cell interactions, response to factors, and the like. The cells are arrayed on a planar or three-dimensional substrate through binding to immobilized or partially diffused probes. Probes of interest include specific binding partners for cell surface molecules, signaling cues that act to regulate cell responses, differentiation factors, etc., which may be arrayed as one or a combination of molecules. The technique can be applied to both adherent and suspension cells. After the cells are arrayed, they may be characterized, or maintained in culture for a period of time sufficient to determine the response to a stimulus of interest.

The methods of the invention allow for passive and active profiling of many cell-surface markers in parallel, programmed patterning of specific cell types, high-throughput stimulation of cells by a variety of immobilized or diffused cues, which may be deposited in any combination and/or concentration, followed by phenotype examination and/or screening, and studies of cell-cell and cell-ECM interactions.

The ability to specifically capture cells onto defined locations at resolutions and feature sizes that are close to cellular dimensions allows for programmed cell patterning and enables close juxtaposition of different cell types, so that their mutual interaction can be examined. These features make the cell microarrays suitable for studying cell-cell and cell-ECM interactions, and for cell migration assays, secretion assays, and active and passive profiling assays. The microarray can optionally be incorporated into a multi-well-based platform by creating arrays within wells (intra-well printing).

### BRIEF DESCRIPTION OF THE DRAWINGS

FIG. 1: Absolute and differential (passive) profiling of suspension cells.

FIG. 2: Specific attachment, profiling and maintenance of adherent cells (human, SVZ-derived, neural and glia progenitors). Specific, ab-mediated (α-human CD58), attachment and maintenance of human neural and glia progenitors derived from neonatal SVZ

FIG. 3: Patterned attachment and maintenance of mouse ES cells.

FIG. 4: Active profiling of suspension cells via functional binding assays (FBAs).

FIG. 5: Active differential profiling of suspension cells.

FIG. 6: A functional binding assay with adherent (mouse ES) cells and discovery of a novel interaction.

FIG. 7: The effects of (i) blocking transcription and (ii) inhibiting LIF signaling on the enhancement of ES cell binding to E-cadherin.

FIG. 8. Spot-directed clustering of mouse ES cells

FIG. 9: Long-term, spot-directed patterning of mouse ES cells on a “sticky” surface.

FIG. 10: “On-slide”, spot-dependent differentiation of mES cells.

FIG. 11: Uncontrolled growth and spontaneous differentiation of mES cells into multiple lineages.

FIG. 12: Enhancement of human Medullo-blastoma cell proliferation by a printed cytokine.

FIG. 13: Spot-dependent changes in differentiation marker profile of human Medullo-blastoma.

### DETAILED DESCRIPTION OF THE EMBODIMENTS

Cell profiling microarrays allow cells to be characterized with respect to their expression of cell surface molecules, and ability to respond to external stimulus in the microenvironment. External stimuli include cell-cell interactions, response to factors, and the like. The cells are arrayed on a planar or three-dimensional substrate through binding to immobilized or partially diffused probes. After the cells are arrayed, they may be characterized, or maintained in culture for a period of time sufficient to determine the response to a stimulus of interest.

Probes of interest include specific binding partners for cell surface molecules, signaling cues that act to regulate cell responses, differentiation factors, etc., which may be arrayed at a range of concentrations, as one or a combination of molecules. Usually each location on a microarray will include at least one probe that is a polypeptide specific binding partner for a cell surface molecule, which may be referred to as a “binding probe”. The “printing” of probes, by which it is intended that a probe molecule is placed on the solid substrate in a specific location and amount, may be used to direct patterned assembly, migration, and programming of multicellular structures. For example, two distinct cell types may be juxtaposed in a specific physical orientation so that their interactions can be systematically observed.

Binding probes of interest include antibodies and fragments thereof, which may bind, for example, cell surface antigens; adhesion molecules; extracellular matrix components; receptor ligands; antigen-bearing MHC constructs; etc. The high affinity and specificity of the binding members lead to a unique cell attachment pattern reflecting the levels of expression of surface antigens. Within certain ranges of cells and binding members, the number of captured cells will be proportional to the expression level of the cognate protein. Differential pre-labeling of different cell populations followed by co-incubation on the slide and multi-color imaging facilitates discrimination of cells based on differences in expression of cell-surface markers.

In addition to a binding probe, probes that generate signals or affect the cell's growth, phenotype, viability, and the like may be used, and can be bound to the microarray substrate, partially diffused on the substrate, present in the medium, etc. Such probes, which may be referred to as “signaling probes”, include a variety of polypeptides and other biologically active molecules, e.g. chemokines, cytokines, growth factors, differentiation factors, drugs, polynucleotides, etc. It will be understood by those of skill in the art that a binding probe may also act as a signaling probe.

By providing for a controlled selection and position of cells, the signals, microenvironments and conditions that provide for a specific phenotype, developmental path, or activation pathway can be explored in a systematic rigorous manner, in specific cell types. Such pathways can include, for example, stimulation of cells by proteins, other environmental cues, direct cell to cell contact, and the like, and may also include two way communication between cells of interest. The arbitrary choice of printed cues allows for reconstruction of well-defined micro-environments that can mimic essential features exhibited by their in-vivo counterparts, thereby serving as simplified model systems for studying their interactions with cells. By controlling the dose of a printed signaling probe, activation and response curves for specific cell types can be mapped out, and the events following activation can be imaged. Systematic mixing of cues might reveal the synergistic structure of a specific process, and possibly some general rules. Likewise, collecting data in parallel from a comprehensive set of defined, naturally occurring signaling cues can potentially lead to a dramatic boost in our understanding of the “language” utilized by cells. Cells of interest include a wide variety of types, each involving a multitude of important processes. for example immune cells activated by antigens, cytokines or other stimulus or that are homing to tissues of interest; developing neurons interacting with glia cells or with vascular cells; embryonic stem (ES) cells progressing through early developmental pathways following fertilization; migrating and differentiating stem cells and cancer cells; cancer cells induced to commit apoptosis; etc.

Cell-microarrays offer advantages over existing multi-well-based approaches for cell stimulation and drug discovery. In multi well plates cells are exposed to globally applied, usually soluble, factors and the cells cannot explore conditions set up in other wells. By contrast, a microarray format supports an open microenvironment, wherein cells are free to move and explore neighboring environments printed on surrounding spots. Combining the open microenvironment concept with much smaller feature sizes makes the cell-microarray format the method of choice for specific cell patterning, and assaying local cell stimulation, migration, secretion, cell-cell and cell-ECM interactions.

In addition, the techniques of the invention offer a higher throughput than existing phenotyping methods, and are faster, simpler and cheaper. Utilizing the ability of cells to respond to exogenous signals, it is a unique tool for cell manipulation, providing for the selective capture of cells and the usage of probe-mediated cell manipulation.

The methods of the invention find use in clinical diagnosis for the profiling and classification of cell samples, e.g. biopsy samples, blood samples, and the like. Stem cell differentiation can be directed or manipulated in specific ways and drugs can be screened for desired phenotypes. In addition, the methods can be used to search for passive and active markers present on cells, e.g. stem cells, cancer cells, etc.

### Definitions

Before the present methods are described, it is to be understood that this invention is not limited to particular methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges, subject to any specifically excluded limit in the stated range. As used herein and in the appended claims, the singular forms “a”, “and”, and “the” include plural referents unless the context clearly dictates otherwise.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

Substrate. Any surface to which the probes of the subject invention are attached, where the probes are attached in a pre-determined spatial array of arbitrary shape. The array may comprise a plurality of different probes, which are patterned in a pre-determined manner, including duplicates of single probe types.

A variety of solid supports or substrates are suitable for the purposes of the invention, including both flexible and rigid substrates. By flexible is meant that the support is capable of being bent, folded or similarly manipulated without breakage. Examples of flexible solid supports include acrylamide, nylon, nitrocellulose, polypropylene, polyester films, such as polyethylene terephthalate, etc. Also included are gels, which allow cells to reside in a three-dimensional environment, while still being completely or partially exposed to potentially immobilized or diffused cues (e.g. collagen gels, matrigels, and ECM gels). Herein, we refer to such realization as a 3D-array. Rigid supports do not readily bend, and include glass, fused silica, quartz; plastics, e.g. polytetrafluoroethylene, polypropylene, polystyrene, polycarbonate, and blends thereof, and the like; metals, e.g. gold, platinum, silver, and the like; etc. In addition, a rigid support may also incorporate a multi-electrode-array for electrical recording and stimulation or any other construct of interest onto which cues could be dispensed.

Derivitized and coated slides are of particular interest. Such slides are commercially available, or may be produced using conventional methods. For example, SuperAldehyde™ substrates contain primary aldehyde groups attached covalently to a glass surface. Coated-slides include films of nitrocellulose (FastSlides™, Schleicher & Schuell), positively-charged nylon membranes (CastSlides™, Schleicher & Schuell), and a polyacrylamide matrix (HydroGel™, Packard Bioscience), etc.

The substrates can take a variety of configurations, including filters, fibers, membranes, beads, particles, dipsticks, sheets, rods, etc., usually a planar or planar three-dimensional geometry is preferred. The materials from which the substrate can be fabricated should ideally exhibit a low level of non-specific binding during binding events, except for specific cases, in which some non-specific binding is preferred.

In one embodiment of the invention, the substrate comprises a planar surface, and the binding members are spotted on the surface in an array. The binding member spots on the substrate can be any convenient shape, but will often be circular, elliptoid, oval or some other analogously curved shape. The local density of the spots on the solid surface can be at least about 500/cm² and usually at least about 1000/cm² but does not exceed about 10,000/cm², and usually does not exceed about 5000/cm². The spot to spot distance (center to center) is usually from about 100 μm to about 200 μm. The spots can be arranged in any convenient pattern across or over the surface of the support, such as in rows and columns so as to form a grid, in a circular pattern, and the like, where generally the pattern of spots will be present in the form of a grid across the surface of the solid support.

The subject substrates can be prepared using any convenient means. One means of preparing the supports is to synthesize the binding members, and then deposit as a spot on the support surface. The binding members can be prepared using any convenient methodology, such as automated solid phase synthesis protocols, monoclonal antibody culture, isolation from serum, recombinant protein technology and like, where such techniques are known in the art. The prepared binding members can then be spotted on the support using any convenient methodology, including manual techniques, e.g. by micro pipette, ink jet, pins, etc., and automated protocols. Of particular interest is the use of an automated spotting device, such as the Beckman Biomek 2000 (Beckman Instruments). A number of contact and non-contact microarray printers are available and may be used to print the binding members on a substrate. For example, non-contact printers are available from Perkin Elmer (BioChip Arrayer™, Packard). Contact printers are commercially available from TeleChem International (ArrayIt™). Non-contact printers are of particular interest because they are more compatible with soft/flexible surfaces.

The total number of binding member spots on the substrate will vary depending on the number of different binding probes and conditions to be explored, as well as the number of control spots, calibrating spots and the like, as may be desired. Generally, the pattern present on the surface of the support will comprise at least about 10 distinct spots, usually at least about 200 distinct spots, and more usually at least about 500 distinct spots, where the number of spots can be as high as 50,000 or higher, but will usually not exceed about 25,000 distinct spots, and more usually will not exceed about 15,000 distinct spots. Each distinct probe composition may be present in duplicate or more (usually, at least 5 replicas) to provide an internal correlation of results. Also, for some tasks (such as stem cell fate manipulation and other cases, in which a group of cells tend to grow and occupy several spots) it is desirable to replicate blocks, each of several identical spots.

By printing onto the surfaces of (preferably flatsurfaced) multi-well plates, one can combine the advantages of the array approach with those of the multi well approach. Since the separation between tips in standard microarrayers is compatible with both a 384 well and 96 well plate, one can simultaneously print each load in several wells. Printing into wells can be done using both contact and non-contact technology, where the latter is also compatible with non-flat multi-well plates.

The amount of binding member present in each spot will be sufficient to provide for adequate binding of cells during the assay in which the array is employed. The spot will usually have an overall circular dimension and the diameter will range from about 10 to 5,000 μm, usually from about 20 to 1000 μm and more usually from about 50 to 500 μm. The binding member will be present in the solution at a concentration of from about 0.0025 to about 10 μg/ml, and may be diluted in series to determine binding curves, etc.

Binding member or Binding Probe. As used herein refers to a member of a binding pair, i.e. two molecules, usually two different molecules, where one of the molecules (i.e., first binding member) through chemical or physical means specifically binds to the other molecule (i.e., second binding member). The complementary members of a specific binding pair are sometimes referred to as a ligand and receptor; or receptor and counter-receptor. For the purposes of the present invention, the two binding members may be known to associate with each other, for example where an assay is directed at detecting compounds that interfere with the association of a known binding pair. Alternatively, candidate compounds suspected of being a binding partner to a compound of interest may be used.

Specific binding pairs of interest include carbohydrates and lectins; complementary nucleotide sequences; peptide ligands and receptor; effector and receptor molecules; hormones and hormone binding protein; enzyme cofactors and enzymes; enzyme inhibitors and enzymes; etc. The specific binding pairs may include analogs, derivatives and fragments of the original specific binding member. For example, a receptor and ligand pair may include peptide fragments, chemically synthesized peptidomimetics, labeled protein, derivatized protein, etc. Polypeptide, glycoproteins, and proteoglycans binding probes are of particular interest, including those found in extracellular matrix. In some embodiments of the invention, the binding probe is a polypeptide other than an antibody or antibody fragment.

Signaling Probe. The signaling probe may be used as an agent for specific cell binding, or may be provided in conjunction with a binding probe. Any molecule capable of eliciting a phenotypic change in a cell may be used as a signaling probe. Signaling probes may be the products of other cell types, (for example, expressed proteins associated with a disease, or secreted in a normal situation or during development), may be compounds associated with the ECM, may be compounds that simulate naturally occurring factors, may be fragments of cells, may be surface membrane proteins free of the membrane or as part of microsomes, etc.

Signaling probes may be used individually or in combination. Illustrative naturally occurring factors include cytokines, chemokines, differentiation factors, growth factors, soluble receptors, hormones, prostaglandins, steroids, etc., that may be isolated from natural sources or produced by recombinant technology or synthesis, compounds that mimic the action of other compounds or cell types, e.g. an antibody which acts like a factor or mimics a factor, such as synthetic drugs that act as ligands for target receptors. For example, in the case of the T cell receptor, the action of an oligopeptide processed from an antigen and presented by an antigen-presenting cell, etc. can be employed. Where a family of related factors are referred to with a single designation, e.g. IL-1, VEGF, IFN, etc., in referring to the single description, any one or some or all of the members of the group are intended. Compounds are found among biomolecules including peptides, saccharides, fatty acids, steroids, purines, pyrimidines, oligonucleotides, polynucleotides, derivatives, structural analogs or combinations thereof.

Signaling probes can include cytokines, chemokines, and other factors, e.g. growth factors, such factors include GM-CSF, G-CSF, M-CSF, TGF, FGF, EGF, TNF-V, GH, corticotropin, melanotropin, ACTH, etc., extracellular matrix components, surface membrane proteins, such as integrins and adhesins, and other components that are expressed by the targeted cells or their surrounding milieu in vivo. Components may also include soluble or immobilized recombinant or purified receptors, or antibodies against receptors or ligand mimetics.

Included are pharmacologically active drugs, genetically active molecules, etc. Compounds of interest include chemotherapeutic agents, anti-inflammatory agents, hormones or hormone antagonists, ion channel modifiers, and neuroactive agents. Exemplary of compounds suitable as binding pair members for this invention are those described in The Pharmacological Basis of Therapeutics, Goodman and Gilman, McGraw-Hill, New York, N.Y., (1993) under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

Cells. Cells for use in the assays of the invention can be an organism, a single cell type derived from an organism, or can be a mixture of cell types, as is typical of in vivo situations, but may be the different cells present in a specific environment, e.g. vessel tissue, liver, spleen, heart muscle, brain tissue, etc.

The invention is suitable for use with any cell type, including primary cells, normal and transformed cell lines, transduced cells and cultured cells, which can be single cell types or cell lines; or combinations thereof. In assays, cultured cells may maintain the ability to respond to stimuli that elicit a response in their naturally occurring counterparts. Cultured cells may have gone through up to five passages or more, sometimes 10 passages or more. These may be derived from all sources, particularly mammalian, and with respect to species, e.g., human, simian, rodent, etc., although other sources of cells may be of interest in some instances, such as plant, fungus, etc.; tissue origin, e.g. heart, lung, liver, brain, vascular, lymph node, spleen, pancreas, thyroid, esophageal, intestine, stomach, thymus, etc.

In addition, cells that have been genetically altered, e.g. by transfection or transduction with recombinant genes or by antisense technology, to provide a gain or loss of genetic function, may be utilized with the invention. Methods for generating genetically modified cells are known in the art, see for example “Current Protocols in Molecular Biology”, Ausubel et al., eds, John Wiley & Sons, New York, N.Y., 2000. The genetic alteration may be a knock-out, usually where homologous recombination results in a deletion that knocks out expression of a targeted gene; or a knock-in, where a genetic sequence not normally present in the cell is stably introduced.

A variety of methods may be used in the present invention to achieve a knock-out, including site-specific recombination, expression of anti-sense or dominant negative mutations, and the like. Knockouts have a partial or complete loss of function in one or both alleles of the endogenous gene in the case of gene targeting. Preferably expression of the targeted gene product is undetectable or insignificant in the cells being analyzed. This may be achieved by introduction of a disruption of the coding sequence, e.g. insertion of one or more stop codons, insertion of a DNA fragment, etc., deletion of coding sequence, substitution of stop codons for coding sequence, etc. In some cases the introduced sequences are ultimately deleted from the genome, leaving a net change to the native sequence.

Different approaches may be used to achieve the “knock-out”. A chromosomal deletion of all or part of the native gene may be induced, including deletions of the non-coding regions, particularly the promoter region, 3′ regulatory sequences, enhancers, or deletions of gene that activate expression of the targeted genes. A functional knock-out may also be achieved by the introduction of an anti-sense construct that blocks expression of the native genes. “Knock-outs” also include conditional knock-outs, for example where alteration of the target gene occurs upon exposure of the animal to a substance that promotes target gene alteration, introduction of an enzyme that promotes recombination at the target gene site (e.g. Cre in the Cre-lox system), or other method for directing the target gene alteration.

A genetic construct may be introduced into tissues or host cells by any number of routes, including calcium phosphate transfection, viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the DNA, then bombarded into cells.

Cell types that can find use in the subject invention include stem and progenitor cells, e.g. embryonic stem cells, hematopoietic stem cells, mesenchymal stem cells, neural crest cells, etc., endothelial cells, muscle cells, myocardial, smooth and skeletal muscle cells, mesenchymal cells, epithelial cells; hematopoietic cells, such as lymphocytes, including T-cells, such as Th1 T cells, Th2 T cells, Th0 T cells, cytotoxic T cells; B cells, pre-B cells, etc.; monocytes; dendritic cells; neutrophils; and macrophages; natural killer cells; mast cells;, etc.; adipocytes, cells involved with particular organs, such as thymus, endocrine glands, pancreas, brain, such as neurons, glia, astrocytes, dendrocytes, etc. and genetically modified cells thereof. Hematopoietic cells may be associated with inflammatory processes, autoimmune diseases, etc., endothelial cells, smooth muscle cells, myocardial cells, etc. may be associated with cardiovascular diseases; almost any type of cell may be associated with neoplasias, such as sarcomas, carcinomas and lymphomas; liver diseases with hepatic cells; kidney diseases with kidney cells; etc.

The cells may also be transformed or neoplastic cells of different types, e.g. carcinomas of different cell origins, lymphomas of different cell types, etc. The American Type Culture Collection (Manassas, Va.) has collected and makes available over 4,000 cell lines from over 150 different species, over 950 cancer cell lines including 700 human cancer cell lines. The National Cancer Institute has compiled clinical, biochemical and molecular data from a large panel of human tumor cell lines, these are available from ATCC or the NCI (Phelps et al. (1996) Journal of Cellular Biochemistry Supplement 24:32-91). Included are different cell lines derived spontaneously, or selected for desired growth or response characteristics from an individual cell line; and may include multiple cell lines derived from a similar tumor type but from distinct patients or sites.

Cells may be non-adherent, e.g. blood cells including monocytes, T cells, B-cells; tumor cells, etc., or adherent cells, e.g. epithelial cells, endothelial cells, neural cells, etc. In order to profile adherent cells, they must be dissociated from the substrate that they are adhered to, and from other cells, in a manner that maintains their ability to recognize and bind to probe molecules. Methods of dissociating cells are known in the art, including protease digestion, etc. Preferably the dissociation methods use enzyme-free dissociation media.

Microenvironment. The cellular microenvironment, or environment, encompasses cells, media, factors, time and temperature. Environments may also include drugs and other compounds, particular atmospheric conditions, pH, salt composition, minerals, etc. Culture of cells is typically performed in a sterile environment, for example, at 37° C. in an incubator containing a humidified 92-95% air/5-8% CO₂ atmosphere. Cell culture may be carried out in nutrient mixtures containing undefined biological fluids such a fetal calf serum, or media which is fully defined and serum free. A variety of culture media are known in the art and commercially available.

Phenotype. Various cellular outputs may be assessed to determine the response of the cells to the input variable, including calcium flux, BrdU incorporation, expression of an endogenous or a transgene reporter, methabolic reporters, electrical activity (e.g. via voltage-sensitive dyes), release of cellular products, cell motility, size, shape, viability and binding, etc. In some case (such as when cells are embedded in a 3D gel), even local pH levels or O₂ and CO₂ concentrations can be assayed. Generally the analysis provides for site specific determination, i.e. the cells that are localized at a spot are analyzed for phenotype in an individual or spot specific manner, which correlates with the spot to which the cells are localized.

The phenotype of the cell in response to a signaling probe or a microenvironment may be detected through changes in cell various aspects, usually through parameters that are quantifiable characteristics of cells. Characteristics may include cell morphology, growth, viability, expression of genes of interest, interaction with other cells, and include changes in quantifiable parameters, parameters that can be accurately measured.

A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. Parameters may provide a quantitative readout, in some instances a semi-quantitative or qualitative result. Readouts may include a single determined value, or may include mean, median value or the variance, etc. Variability is expected and a range of values for each of the set of test parameters will be obtained using standard statistical methods with a common statistical method used to provide single values.

Parameters of interest include detection of cytoplasmic, cell surface or secreted biomolecules, frequently biopolymers, e.g. polypeptides, polysaccharides, polynucleotides, lipids, etc. Cell surface and secreted molecules are a useful parameter type as these mediate cell communication and cell effector responses and can be readily assayed. In one embodiment, parameters include specific epitopes. Epitopes are frequently identified using specific monoclonal antibodies or receptor probes. In some cases the molecular entities comprising the epitope are from two or more substances and comprise a defined structure; examples include combinatorially determined epitopes associated with heterodimeric integrins. A parameter may be detection of a specifically modified protein or oligosaccharide, e.g. a phosphorylated protein, such as a STAT transcriptional protein; or sulfated oligosaccharide, or such as the carbohydrate structure Sialyl Lewis x, a selectin ligand.

A parameter may be defined by a specific monoclonal antibody or a ligand or receptor binding determinant. Parameters may include the presence of cell surface molecules such as CD antigens (CD1-CD247), cell adhesion molecules including integrins, selectin ligands, such as CLA and Sialyl Lewis x, and extracellular matrix components. Parameters may also include the presence of secreted products such as lymphokines, chemokines, etc., including IL-2, IL-4, IL-6, growth factors, etc.

### Profiling Methods

Passive Profiling. In methods of passive profiling, a suspension of cells, which may be adherent cells or non-adherent cells, is allowed to bind to a microarray of binding probe molecules. The population of suspended cells, as described above, is added to a microarray comprising bound probes. The suspension is applied to the substrate without a cover or under a coverslip, or into a fixed volume of “hybridization” or “staining” or a “perfusion” chamber. In a passive profiling assay, a probe could be any type of molecule capable of sufficiently strong and specific interaction with cells. In one embodiment, the probe is an antibody or fragment thereof. In another embodiment, the probe is a polypeptide other than an antibody, including cell adhesion molecules (CAMS) and extracellular matrix (ECM) components, e.g. laminin, fibronectin, collagen, vitronectin, tenascin, restrictin, hyaluronic acid, etc. cytokines; growth factors; and the like. The incubation time should be sufficient for cells to bind the probes. Generally, from about 4 minutes to 1 hr is sufficient, usually 20 minutes sufficing.

While many assays are performed with live cells, passive assays may also be performed with fixed cells. Cells fixed with various concentrations of reagents such as PFA, glutaraldehyde, methanol, acetic acid, etc. can be used alone, or in comparison with non-fixed cells.

After incubation, the insoluble support is generally washed to remove non-specifically bound cells in any medium that maintains the viability of the cells and the specificity of binding, e.g. DMEM, Iscove's medium, PBS (with Ca⁺⁺ and Mg⁺⁺), etc. The number of washes should be determined experimentally for each application and cell type by observing the degree of non-specific binding following each wash round. Usually from one to six washes would be employed, with sufficient volume to thoroughly wash non-specifically bound cells present in the sample.

Passive profiles can be absolute or differential. In an absolute profile, a single cell type is added to the microarray, and the number of bound cells detected. Occupied spots denote the presence of the corresponding cell surface marker to the binding probe. Over a range of cell and probe concentrations, the higher the expression level, the higher the number of captured cells. However, absolute profiles can be susceptible to spot- and slide-related variations.

A differential profile is a competitive assay, where two or more cell types/populations are pre-labeled with different labels, combined and applied to a single slide, where they compete for binding to probe molecules. Following washout, the slide can be scanned and scored for the relative number of label present for each of the cell types.

In order to detect the presence of bound cells from each type, a variety of methods may be used. In an absolute assay, the cells need not be labeled at all or may be labeled with a detectable label, and the amount of bound label directly measured In a differential assay, labeled cells may be mixed with differentially labeled, or unlabeled cells and the readout could be based either on the relative number of pixels with a given label (or no label, respectively) or the relative number of cells with a given label (or no label, respectively). In yet another embodiment, the cells themselves are not labeled, butcell-type-specific second stage labeled reagents are added in order to quantitate the relative number of cells from each type, or to phenotype the cells. In some instances the cells will not be quantitatively measure, but will be observed for such phenotypic variation as morphology, adherence, etc.

Examples of labels that permit direct measurement of bound cells include radiolabels, such as ³H or ¹²⁵I, fluorescers, dyes, beads, chemilumninescers, colloidal particles, and the like. Suitable fluorescent dyes are known in the art, including fluorescein isothiocyanate (FITC); rhodamine and rhodamine derivatives; Texas Red; phycoerythrin; allophycocyanin; 6-carboxyfluorescein (6-FAM); 2′,7′-dimethoxy-4′,5′-dichloro-6-carboxyfluorescein (JOE); 6-carboxy-X-rhodamine (ROX); 6-carboxy-2′,4′,7′,4,7-hexachlorofluorescein (HEX); 5-carboxyfluorescein (5-FAM); N,N,N′,N′-tetramethyl-6-carboxyrhodamine (TAMRA); sulfonated rhodamine; Cy3; Cy5; etc.

Cell microarrays can be scanned to detect binding of the cells, e.g. by using a simple light microscopy, scanning laser microscope, by fluorimetry, a modified ELISA plate reader, etc. For example, a scanning laser microscope may perform a separate scan, using the appropriate excitation line, for each of the fluorophores used. The digital images generated from the scan are then combined for subsequent analysis. For any particular array element, the ratio of the fluorescent signal with one label is compared to the fluorescent signal from the other label DNA, and the relative abundance determined.

A specific passive profile of interest is the analysis of T cells. Arrays of MHC monomers, tetramers, peptide-loaded DimerX (BD-Pharmingen), etc. that provide MHC presentation of antigens can be microarrayed for direct, high-throughput diagnosis/analysis of antigen-specific T cells. Peptide-bearing constructs can be printed on a substrate and bound to a T cell sample of interest. Slowly circulating the sample over the printed region (e.g. using a low flow peristaltic pump and a sealed incubation chamber with inlet and outlet, such as the CoverWell™ perfusion chambers from Grace Biolabs) may increase the sensitivity by giving rare populations of antigen-specific T cells more chances to find targets on the surface. Other means to increase the sensitivity would be to employ a templated chamber to guide the flow along the different antigen-bearing constructs and/or to increase the number of identical spots of each of the constructs, in a direction that is perpendicular to the direction of flow.

Active profiling (AP) and functional binding assays (FBA). In an AP assay, the presence of a given marker is indirectly detected by assaying the fingerprints of its activation. An FBA is a specific type of AP, in which a printed cue (signaling probe) actively induces cells to bind to a co-spotted cue (binding probe). In this case, the presence of the receptor involved in the activation is assayed by the induction or enhancement of cell binding. FBA can be used to screen for cues capable of enhancing cell binding to a particular ECM component or CAM; for ECMs and CAMs to which cells can bind following the activation by a specific cue.

Similarly to passive profiling, functional binding assays can be performed in an absolute or a differential manner. However, unlike passive profiling, the binding probe in a functional binding assay is either co-spotted with an additional, stimulating cue or juxtaposed to a stimulating cue (e.g. the latter will be present on an adjacent spot). Other examples of active profiling, which do not necessarily involve the induction or enhancement of binding, include any assayable change in one or more cell parameters on spots that contain a given signaling probe, vs. those spots that that do not contain that signaling probe. For example, the presence of a specific growth factor receptor can be inferred from a reproducible increase in cell proliferation only on spots that contain the corresponding growth factor.

It will be understood by those of skill in the art that some binding probes also elicit a cellular response. Even antibodies may be effectively used in the context of an active profiling assay if binding stimulates or blocks a receptor or other marker in a manner that can be detected with another reporter. For example, T cells may be stimulated by co-printed CD3 and CD28, followed by up-regulation of CD69, which can then be detected by immunostaining of cells on combined CD3 and CD28 spots vs. just CD3 or just CD28 spots. In this case, up-regulation of CD69 on the combined spots would indicate the presence of both CD3 and CD28 on the cell surface, even when the level of one of the two markers (say CD28) does not suffice to capture the cells on the corresponding antibody (in which case, the cells would only bind the combined and the CD3 spots, and the CD69 up-regulation would refer only to the combined vs. CD3 spots).

A signaling probe can be detected for its ability to enhance the binding of cells to a particular binding probe, and/or for other changes in phenotype. For example, a signaling probe may induce expression of a cell surface marker. While the starting cell population will be unable to bind to the counterpart binding probe, cells responding to the signaling probe will bind.

Results of active profiling assays can be read out as the absolute or differential scores. Readouts of interest include calcium flux following stimulation, changes in expression of markers including reporter genes, and cell surface receptors, changes in BrdU incorporation corresponding to changes in proliferation rates, pulses of voltage sensitive dyes following the induction of electrical activity, changes in cell motility, etc.

One embodiment of active profiling assays is screening for activity of drug candidates, by printing with or without a capture molecule. Candidate agents include agents that act inside the cells, and on the cell surface, as described above. To improve the interactions with cells, candidate agents may be printed onto a film-coated slide or in a 3D gel. Sustained release of an agent can be achieved by printing a mixture that releases active agents from a polymer gel or by slow hydrolysis of a linker, through which the active agent is connected to the surface.

In some embodiments, the candidate agent is bound to a polypeptide carrier, which may be a binding probe, a receptor that specifically interacts with the agent, and the like. For example, steroid compounds may be presented in conjunction with their appropriate carrier protein, e.g. retinol binding protein, corticosteroid binding protein, thyroxin binding protein, etc.

Included in the candidate agents that may be screened are arrays of peptide libraries. Peptides, which may provide signaling and/or binding functions, are tested by exposing cells to an arrayed library, which may be random sequences, shuffled sequences, known sequences that are randomly mutated, etc. Reactive side chains may be capped prior to the immobilization and uncapped just before applying the cells. The peptides can be bound to the substrate directly, or via a linker attached to one end, bound to a carrier protein, etc. The peptides may be synthesized directly onto the substrate, (see, for example, U.S. Pat. No. 5,143,854).

Migration assays. An aspect of active profiling is a migration assay. In a migration assay putative chemo-attractant cues are printed next to and/or together with a capture molecule. The migration of cells is detected, and compounds scored for their ability to direct such migration. In one embodiment, the directed movement of cells toward nearby chemokine-containing spots, e.g. SDF-1; and/or up a gradient of a chemokine. Such a gradient can be set by increasing the chemokine concentration from spot to spot and/or printing on a substrate that supports the diffusion of printed proteins (e.g. a commercially available collagen gel such as “VITROGEN 100”). The chemokine may or may not be printed with a capture moiety. Also, the cells can either be specifically immobilized with a binding probe, or could be grown un-patterned within a 3 dimensional gel, that is later printed with chemokine fields.

Another embodiment for high-throughput migration assays places cells of interest on top of two ECM gel layers, where the top layer is very thin, having a thickness of from about 0.05 to about 0.2 mm, and the bottom layer is thicker, having a thickness of from about 3 mm to about 5 mm. A 3D array of candidate chemoattractants is printed on one of the layers, and the migration of cells across the layers in response to diffusing chemoattractants is scored. Where there is upward diffusion of chemoattractants would stimulate downward cell migration. Down-migrating cells would cross over to the bottom layer, and the chemotactic activity of each factor is scored by the number of crossing over cells in the portion corresponding to that factor. Alternatively, the cells are placed cells below an empty thin layer, which in turn lies below the printed thick layer. The thin layer may also be replaced with any other layer that can be traversed by cells that are responding to chemotactic agents (for example, transwell filters that are commonly used in standard migration assays).

Migration and spreading of cells out of the printed regions are associated with secretion of ECM components that can be required for attachment and migration. Such secretions can be locally analyzed by standard immuno-staining against specific components that may be secreted.

Cell-cell interaction assays. The ability to specifically capture any type of cells onto defined locations and to form patterned surfaces with feature sizes on the order of one or few cell diameters, can be used to juxtapose two or more different cell types, and study their mutual interactions. Different cells can be immobilized within the same spots by printing a common binding probe or co-printing of two or more cell-type specific binding probes. Alternatively the cells can be immobilized separate, nearby spots using cell-type-specific binding probes. If cell-type-specific capture molecules are not known, the cells can be screened in an absolute or differential profiling experiment to determine suitable binding partners.

In order to obtain juxtaposition of distinct cell types on nearby spots, those populations may need to be segregated, such that each spot will include only one cell type. This can be achieved by performing an initial screen of cell-type-specific binding partners to screen for binding probes that segregate these populations (as judged by morphology, marker profile, or any other suitable method). For example, one can segregate a mixture of neural and vascular progenitors by exposing the cells to an antibody array that includes a set of antibodies against putatively unique endothelial markers and another set for neuronal/glial-specific markers. The slide can then be simultaneously stained with at least one antibody from each set, to find binding probes within these sets that provide optimal segregation. These binding probes are then be printed at the desired pattern on another array, and thus used for simultaneous segregation and juxtaposition of neural and endothelial progenitors. Subsequently, the cells can be co-cultured and the juxtaposed cells can be compared to non-juxtaposed cells that were captured and cultured on the same slide. An alternative approach can print different cell types onto nearby spots using a non-contact printing technology.

Another specific profile of interest, which may be a passive or an active profile, involves delayed cell patterning. In such cases, they cells do not immediately bind to the binding probes, but when maintained in culture for a period of time, e.g. about 12 hours, 24 hours, or over several days, over time will come to bind to the spots. This may be due to changes in the cell phenotype, e.g. in response to local environment, or due to low level binding. Delayed patterning can also occur either on a non-specifically reactive surface or within ECM gel arrays, wherein the cells are cultured in the gel prior to the printing, and/or when cells are dispensed in the vicinity of already printed cues.

Cell-fate manipulation. In one aspect, active profiling detects the effects of an agent on cell differentiation. Cells suitable for such assays include a variety of progenitor and stem cells. Stem cells of interest include hematopoietic stem cells and progenitor cells derived therefrom (U.S. Pat. No. 5,061,620); neural crest stem cells (see Morrison et a/. (1999) Cell 96:737-749); embryonic stem (ES) cells; mesenchymal stem cells; mesodermal stem cells; etc. Other hematopoietic “progenitor” cells of interest include cells dedicated to lymphoid lineages, e.g. immature T cell and B cell populations. Progenitor cells have also been defined for liver, neural cells, pancreatic cells, etc. Profiling may screen molecules that can direct differentiation, de-differentiation and trans-differentiation events. In particular, the control over ES cell differentiation is especially important for both regenerative medicine and for understanding the very early stages of mammalian development. A common theme in development is the influence of local morphogens on cell-fate decisions. The methods of the invention provides means of rigorously and systematically exploring the actions of concentrated purified morphogens (e.g. Notch, BMP-4, Wnt-1, bFGF, Shh, their modified forms, other members of their families, etc) by constructing local (discrete or continuous) gradients and fields thereof, to which the cells of interest can be exposed and then profiled. It can also be used to examine the effects of their immobilization, association with matrix components or mixtures, or with one another.

Local effects can be obtained by immobilized (membrane-bound and/or ECM-bound) signaling probes; high local concentrations of secreted cues from adjacent cells; differential cell response to different concentrations of a signaling probe; to combinations of signaling probes; and the like. The cell microarray platform offers a unique opportunity to mimic those scenarios in a very high-throughput manner. Thus, for example, fields of immobilized or diffused morphogens, e.g. Shh, FGFs, Wnts, Notch, TGFs etc., and many other cytokines/growth factors/hormones can be deposited at arbitrary combinations and concentrations, usually in combination with a binding probe, e.g. CAM, ECM component, etc. Alternatively, the stem or progenitor cells may be embedded in a three-dimensional matrix (described in more detail below), where the use of a binding probe is not necessary.

Additional factors that can be deposited on the microarray are conditioned mediums, and cell fragments. Undifferentiated ES cells can be cultured on such arrays and can be screened for spot (bound) and medium (unbound) conditions required for the appearance of a desired differentiation phenotype. The latter can be detected as a morphological feature, e.g. the appearance of elaborate neuronal processes in the case of neuronal differentiation, cell contractions for myocytes, etc.; by a lineage-controlled reporter gene; staining with a set of lineage restricted markers; and any of the standard readouts that are used to phenotype cultured cells.

Both the morphological and lineage-controlled reporter gene readouts can be continuously monitored in real time and/or recorded time-lapse using commercially available systems for live cell recording that have scanning capabilities and are equipped with a proper environment control system (e.g. the Axon Instruments “ImageXress” system).

In addition to the above described formats, the assays of the invention may use three dimensional gels, e.g. an ECM gel such as “VITROGEN 100” collagen gel, (Cohesion Technologies, Inc). The probes may be printed on the gel within which cells are pre-embedded; signaling probes may be printed together with binding probes, or followed by exposure to the cells and washout of non-attached cells. Alternatively the cells may be printed together with signaling probes (provided that the gel is properly hydrated).

Printing onto gels can be performed with a non-contact micro-dispensing system, e.g. Packard Bioscience “Biochip Arrayer”. Such systems utilize a non-violent dispensing mechanism (contraction of piezzo-electric sleeve). Tips with a relatively wide open, e.g. at least about 75 μm, that provide for drops of a volume of greater than 300 nl. volume of each dispensed drop (0.350 nL), allow for cell deposition along with signaling probes of interest. A positioning camera can allow probes and cells to be locally added at later stages.

The three dimensional array and some film coated slides as substrates for printing allows for diffusion of signaling probes, where the effect of a gradient on a cell can be analyzed. The printed probes diffuse and form potentially important continuous gradients.

ES cells can be applied and washed away from the surface of an un-printed “VITROGEN” collagen gel, or can be cultured within it by mixing them with the neutralized liquid phase of the gel prior to gelation (fibrillogenesis), initiating gelation by raising the temperature from 4° C. to 37° C., and culturing the (solid) gel in a standard ES medium.

The agents utilized in the methods of the invention may be provided in a kit, which kit may further include instructions for use. Such a kit may comprise a printed microarray. The kit may further comprise cells, assay reagents for monitoring changes in cell phenotype, singling probes, and the like.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### Experimental

### EXAMPLE 1

### Preparation of Cell Profiling Array

A protein microarray was assembled, using different binding and signaling probes, where the binding probes are proteins capable of strong and specific binding to molecules present on the cell surface and signaling probes are proteins capable of actively inducing or enhancing the binding of the cells to the binding probes. Note that the usage of signaling probes far exceeds the scope of profiling assays and that binding probes can be found to function as signaling probes. Cells are then incubated on the array to provide for specific binding and spatial distribution of the cells.

Methods

Array preparation: Solutions of the following proteins were prepared: laminin, fibronectin, collagen, gelatin vitronectin, tenascin, restrictin, chondroitin sulfate, hyaluronic acid, a mixture of all these ECM components, rhICAM-1, rhVCAM-1, mixture of rhSDF-1α and rhVCAM-1, anti-human CD2, CD3, CD4, CD28, CD45, CD58, CD44, CD29, CD95L, CD104, CD123, anti-CCR7, anti-α₄β₇ integrin, β-NGF and bFGF at concentrations ranging from 0.01 μg/μl and 0.5 μg/μl all diluted in a PBS buffer without glycerol (addition of glycerol to the spotting solution slows down the drying of the spots and could potentially improve the stability of some printed proteins). The proteins were spotted onto SuperAldehye glass slides.

For the protein-cell-array work, the SuperAldehyde slides usually work well without any pre-printing or post-printing processes (though some cell types that tend to stick nonspecifically to the SuperAldehyde slide can require post-print blocking with PBS+1-2% BSA). Also, to ensure the removal of unbound probe the slides can be washed in three times for 30′ in PBS+0.5% Tween20. The HydroGel slides require, in addition, preprocessing to remove the storage agent present in the substrate (as well as to ensure consistent, uniform substrate condition), and post-processing to immobilize the proteins. Pre- and post-processing of the HydroGel slides was performed as described in the HydroGel protocol guide.

The proteins were prepared in a 384-well microtitre plate. The proteins on a single array may be the same or different depending on the printing plan. Printing was performed with 8- to 32tip print head depending mostly on the desired print area (which, in turn, is limited by the amount of available cells), but also on the number of different samples to print. The typical local density of the printed spots was 3265/cm² (spot to spot distance of 175 μm) and the maximal density is 4444/cm² (1501 μm). The arrays were sealed in an airtight container. They can be stored at 4° C. for short term storage (˜1-2 month) or frozen for longer storage.

The back side of the slides was marked with a diamond scribe or indelible marker to delineate the location of groups of spots. In some cases, printed Cy5-conjugated BSA (at 0.2 μg/μl) and positive control spots (to which the cells were known to bind at high numbers), were used as coordinate systems.

### EXAMPLE 2

### Profiling of Suspension Cells

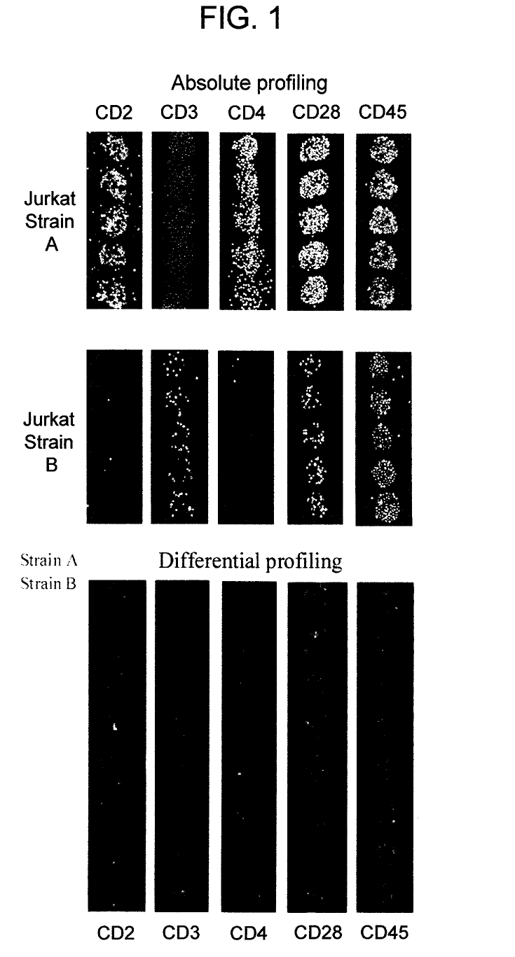

Absolute and differential (passive) profiling of suspension cells. Two different schemes for passive profiling are used, as shown in FIG. 1, absolute and differential. In absolute profiling (upper two panels), each cell type was incubated separately on a slide printed with an array of “probe” molecules: CD2, CD3, CD4, CD28 and CD45. Following washout of non-specific binding, the slide is imaged with simple light microscopy and each spotted region is scored for the number of attached cells. Occupied spots indicate specific binding of the cell sample to the probe present on the substrate, i.e. the cells express the cognate receptor, or specific binding partner of the probe. Over a range of cell and probe concentrations, the number of captured cells is proportional to the level of expression of the specific binding partner. The absolute scheme is sensitive to non-uniform printing, quality of the probe molecule and slide treatment.

Differential profiling reduces spot- and slide-related variations. Two or more cell types/populations are pre-labeled with different dye markers. The two populations are combined and applied to a single slide, where they compete on the same probe molecules. Following washout, the slides were scanned with a commercial fluorescent scanner and scored for the relative number of pixels of one color versus the other colors.

Shown in FIG. 1 are absolute (top panel) and differential (bottom panel) profiling of two different strains of Jurkat cells and 5 probe molecules (antibodies against human CD antigens, commercially available from BD-Pharmingen), printed at a concentration of 0.25 μg/μl in PBS solution with 0.09% (w/v) sodium azide (compatible with aldehyde slides and HydroGel-coated slide) on aldehyde slides (Telechem “SuperAldehyde”). In each case, the cells were suspended in RPMI+1% BSA, incubated in triplicates (i.e. on 3 separate slides) for 10′ at 37C and 5% CO₂ but without a cover or an incubation chamber, and washed by gently moving each slide back and forth 3-5 times in a large chamber of PBS at room temperature.

In the differential profiling experiment (colored images), the two different cell populations were pre-labeled with Cy3 and Cy5 dyes that react non-specifically with free amine groups (Amersham, mono-reactive CyDye). To label the cells, dry aliquots (1 tenth of supplied dye vial) of each of the dyes (Cy3 and Cy5) were re-suspended in 1% of DDW and added to 250 μl of cells (10⁷ cells/ml) in CMF PBS (37° C). The labeling reaction was performed for 10′ in the incubator, following which the cells were washed twice with CMF PBS, and re-suspended in RMPI containing 1% BSA. The concentration of Cy3- and Cy5-labeled cells were matched and two aliquots were then pulled and applied to the slide (in triplicate). Following the removal of non-specific binding, the slide was dried by 2.5′ of centrifugation at 600 rpm (Beckman CS-6R centrifuge) and scanned at 5 μm resolution in a standard fluorescent slide scanner (Axon Instruments “GenePix 4000B”).

In both the absolute and differential assays, the attachment patterns were strain-specific, reproducible, and consistent with each other.

### EXAMPLE 3

### Attachment and Profiling of Adherent Neural and Glial Progenitor Cells

The use of cell array profiling with adherent cells requires an initial dissociation of the cells without compromising their ability to recognize and bind the probe molecules. Enzyme-free dissociation of cells (e.g. using Specialty Media, “Enzyme Free dissociation solution, PBS based”) has been shown to be effective in all cell types tested, including a primary culture of human neural/glial progenitors, a human medullo-blastoma brain tumor cell line, and mouse embryonic stem (mES) cells. The profiling method was also used with fairly large cells. For example, the diameter of neural progenitors that become spread on the slide can be of the order of the spot diameter. But shortly after the dissociation, the cells are round and compact enough so that a single spot can accommodate for over ten cells.

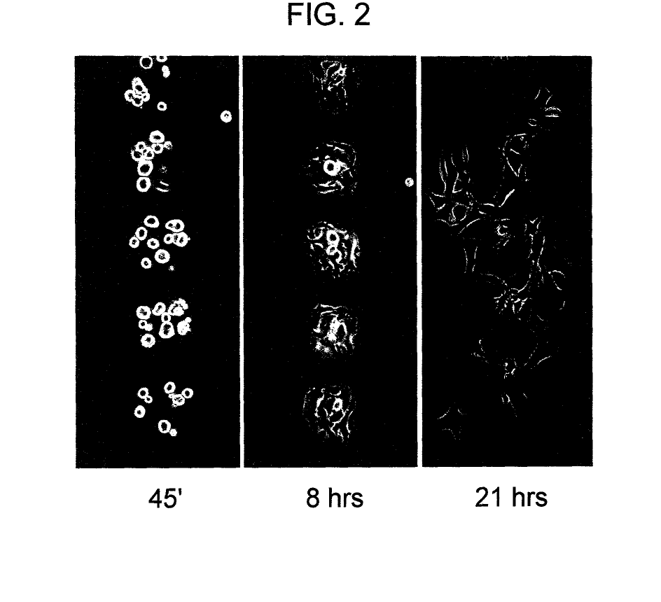

FIG. 2 shows passive profiling of large monolayer cells without compromising long-term viability and motility of the captured cells. Approximately 5×10⁵ human (neonatal) glia/neural progenitor cells were dissociated non-enzymatically for 5′ using an enzyme-free dissociation solution (Specialty Media), re-suspended in DMEM/F12/PS+10% BIT, and incubated on a “SuperAldehyde slide” (TeleChem Inc.) slide printed with several antibodies, cell adhesion molecules (CAMs) and extracellular matrix (ECM) components. Inter-spot separation is 175 μm. Following a 20′ incubation period, the slide was washed in a DMEM chamber to remove non-specific binding and re-incubated for 48 hours in a well-defined medium (DMEM/F12/PSF+10% BIT+20 ng/ml EGF and bFGF).

Shown in FIG. 2 are three time points of a single portion of the array, containing 5 spots of anti-human CD58 (LFA-3). During this time, the cells spread, migrated and acquired more differentiated morphology. This shows that specific attachment of the cells via antibodies does not interfere with their ability to stay viable and healthy, move around, explore their surroundings (including nearby spots), differentiate, and secrete ECM components.

These results demonstrate the feasibility of cell-cell interaction assays that are based on specific cell patterning. They also bring to mind other types of assays, such as migration assays (e.g. cells moving towards a chemoattractant printed onto a nearby spot), and secretion assays (wherein the slide is stained for particular ECM components and/or other substances that might be secreted by a given cell type).

### EXAMPLE 4

### Patterned Attachment and Maintenance of Mouse ES Cells

Non-enzymatically dissociated R1 mouse ES cells were immobilized on various antibodies, ECM components and CAMs, without compromising long-term viability and their ability to divide onto the spots, even when captured via 0.25 μg/μL anti-mouse CD29 (anti-Integrin β₁).

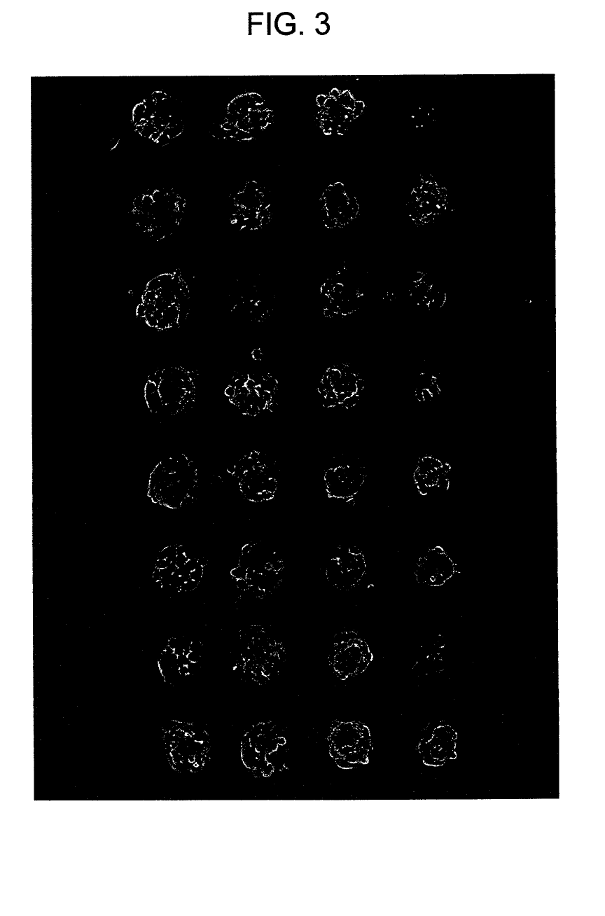

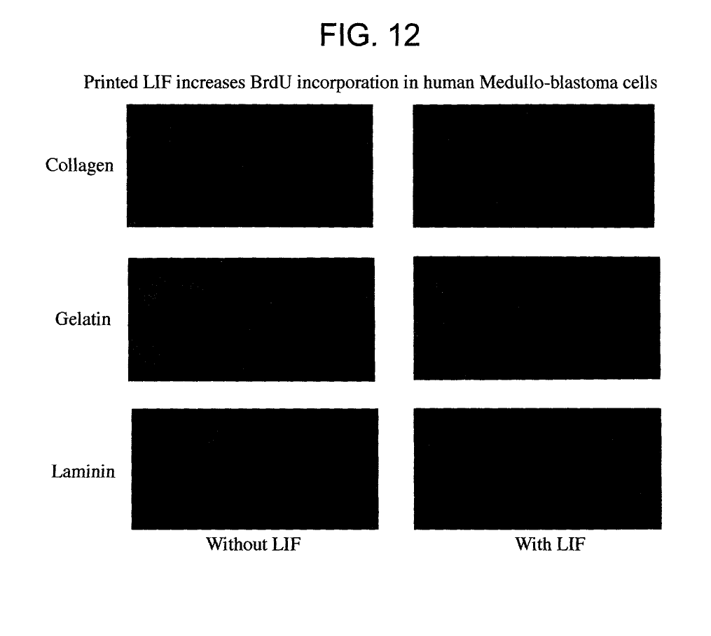

Shown in FIG. 3 are 8 by 4 ES clusters bound to identical spots containing a 4:1 mixture of laminin (0.59 μg/μl) and LIF (10 μg/ml). The picture was taken 21 hours after the initial cell arrest on the slide. Inter-spot separation is 175 μm. Undifferentiated mES cells were dissociated for 15′ with enzyme-free dissociation solution (Specialty Media), resuspended in DMEM/F12/PS+10% BIT, incubated on a “SuperAldehyde slide” (Telechem Inc.) for 20′, and washed in a big chamber of DMEM to remove non-specific binding to the slide. The slide was then transferred into commercially available serum-free medium (Gibco “Knockout DMEM”+15% “Knockout Serum Replacement”+1×non-essential amino acids+1×L-glutamine+1×PS), and the cells were cultured in it for 2 weeks, during which the clusters grew, connected to form a huge clump of relatively undifferentiated cells that eventually gave rise to differentiated cells (example of these later stages is shown in FIG. 12).

### EXAMPLE 5

### Active Profiling of Suspension Cells via Functional Binding Assays

Functional binding assays (FBA) are a type of profiling assay based on receptor activation. This type of assay is useful for studying cell-ECM interactions in a very detailed and systematic manner. It is also useful to detect markers that cannot be detected in a standard passive assay, for example when there is no available antibody, or when expression levels of the binding partner are too low). An FBA tests the ability of a cue of interest to enhance cell binding to a particular ECM component and/or a CAM. This is done by exposing the cells of interest to varying concentrations of a CAM and/or ECM component, which is co-printed on an array with varying concentrations of a (signaling) cue of interest. Microarray technology enables the examination of many CAM-cue pairs on a single slide.

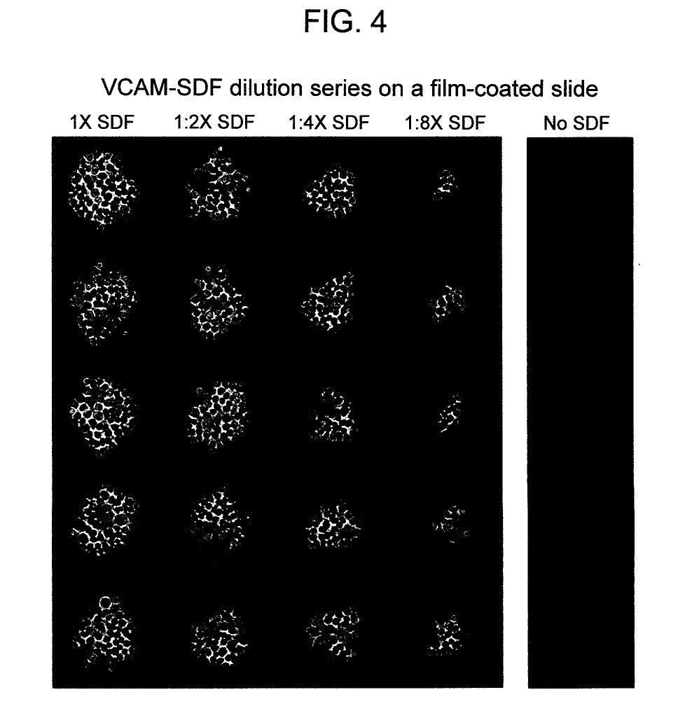

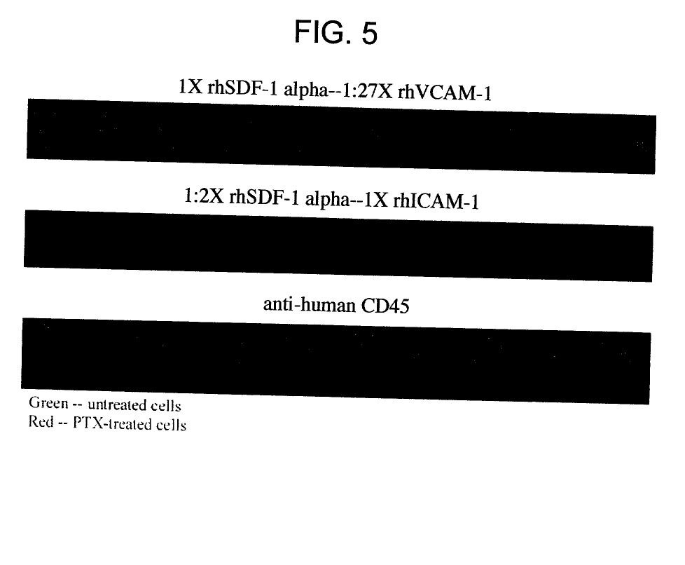

Similarly to passive profiling, active profiling via FBA can be performed in an absolute or differential manner (as shown in FIGS. 4 and 5, respectively). The FBA in FIG. 4 shows a dose-dependent binding of Jurkat T cells to a cell adhesion molecule (rhVCAM-1, R&D Systems) co-spotted with a dilution series of a chemokine (rhSDF-1α, R&D Systems). The SDF activates cell surface integrins on the T cells, thus enhancing their binding to the printed VCAM (see also FIG. 5). Shown in FIG. 4 are cell clusters attached to a 5 by 4 array of spots comprising a given concentration of VCAM (0.055 μg/μl), co-spotted with 2-fold SDF dilutions or without SDF (inter-spot separation is 175 μm). Spots with just SDF were also represented on the slides but no cells were found on those. Each column corresponds to a different SDF dilution, where 1×SDF=37.5 μM (supplied in RPMI containing 3% serum). Note that since the VCAM and SDF are co-spotted at a 1:1 ratio (2.5 μl of each are loaded in the printed well) their final concentrations are half that specified above (i.e. 0.0275 μg/μl, and 18.75 μM, respectively).

In this example, the SDF and VCAM were deposited onto a slide coated with a thin film of polyacrylamide (“HydroGel” slide, Packard Biosciences) using a standard contact printing technology. The slide was pre-processed prior to printing and post-processed according to the manufacturer instructions (except for washing with PBS+0.5% Tween following 16 hrs of probe immobilization). Usually, the “HydroGel slides” give rise to lower non-specific binding (i.e. lower background) as compared with the “SuperAldehyde” slides, but the sensitivity is also lower. In addition, the cells spread better on the “SuperAldehyde” slides and remain attached for longer time periods. The choice of a substrate is task-dependent. Other, commercially available film substrates are the Nitrocellulose-coated “FastSlides” and the positively-charged-nylon CastSlides” (both from Schleicher & Schuell).

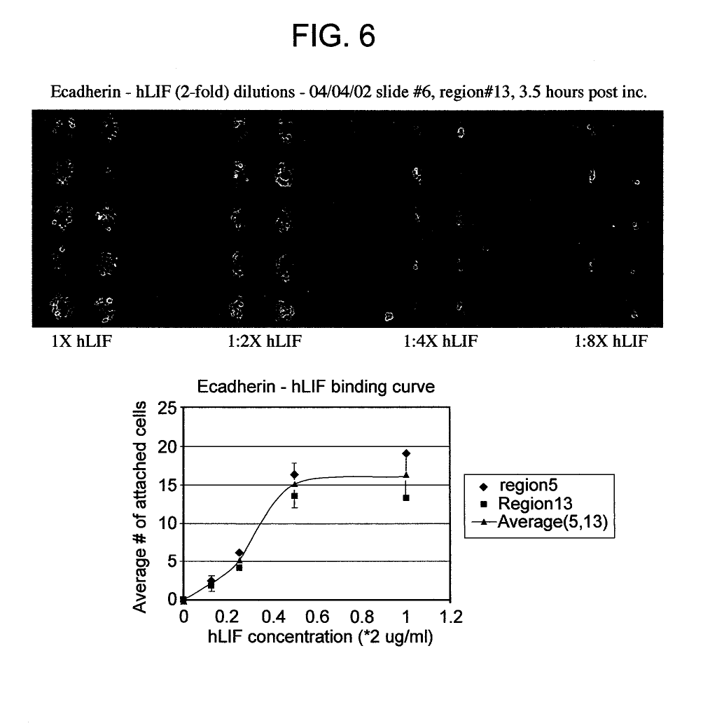

The cells were re-suspended in RPMI+1% BSA, applied to the slide for 6′, gently washed in a chamber of slightly warmed RPMI, and fixed with 4% PFA (4C). The choice of SDF and VCAM concentrations depends primarily on the type and density of cells as well as the task at hand. For example, to construct a binding curve for Jurkat cells, typical ranges should be 0.01-0.1 μg/μl of VCAM, and 0.2-40 μM SDF (higher VCAM doses would lead to SDF-independent binding). A binding curve relates the number of attached cells to the concentration of at least one of the spotted reagents (for an example, see FIG. 6). Within these ranges, the binding is a monotonically increasing function of both the SDF and VCAM. When the Jurkat cells are captured by VCAM spots on a SuperAldehyde slide, without fixation, and incubated in RPMI+15% FBS+1×PS, the cells keep on proliferating and constantly release clones of themselves into the solution while the spots remain occupied with bound cells (care should be taken not to shake the culture dish too vigorously, as that could lead to detachment of the bound cells). Starting with as low as few hundred cells on the spots we found dishes with millions of cells after 1-2 weeks.

A differential functional assay is shown in FIG. 5. The data reveal differential response to spotted SDF-1α signal. Mixtures of SDF and either rhVCAM-1 (upper panel) or rhICAM-1 (middle panel) were printed on the same slide (at 10 replicas per mixture), along with positive-control antibody spots (bottom panel). The slide was incubated for 6′ with a pulled population of Pertussis-Toxin-treated (red) and untreated control Jurkat T cells (green) that were pre-labeled with Cy5 and Cy3, respectively. Treated cells were pre-incubated for 5 hours with 200 ng/ml of Pertussis Toxin (PTx), available from LIST BIOLOGICAL LABORATORIES, INC.

After a gentle wash in RPMI, the bound cells were fixed for 15′ with 4% PFA (at 4C), washed again in PBS for 10′, centrifuge-dried and scanned at 5 μm resolution. Shown in each field are 10 cell clusters attached to identical spots (175 μm interspot separation) comprising a given dilution of SDF and VCAM or SDF and ICAM (1×SDF 1×ICAM, and 1:27×VCAM are final concentrations of 18.75 μM, 0.25 μg/μl, and 0.0275 μg/μl, respectively). The lowest panel serves as a positive control attachment of both cell populations to (0.5 μg/μl) anti-CD45 (BD-Pharmingen). Pre-treatment of cells with PTx induces inactivation of Gi proteins, and consequently, interferes with SDF-1α enhancement of T cell binding to VCAM via activation of cell surface integrins. If an “on-spot” SDF signaling takes place on the slide, the untreated cells are expected to out-compete the treated ones on the SDF-containing spots (more red than green pixels), as is shown in the two upper panels of FIG. 5.

By contrast, on the positive control (CD45) spots, red and green pixels are almost equally represented, indicating that the resulting differential binding to the SDF-containing spots is not due to a significant difference in the number of red vs. green cells. Such control spots can be used to normalize the results for relative differences in cells representation. Thus, this experiment clearly supports the involvement of immobilized SDF in integrin activation and subsequently, cell arrest.

Chemokine-mediated integrin activation followed by cell arrest are two essential steps in lymphocyte trafficking in vivo. The latter is guided by chemokine and adhesion cues presented by the cells of the vascular endothelium. The above functional binding assay mimics these steps by “playing the role” of the endothelial wall in presenting the cues. Note that the in vivo process of integrin activation followed by cell arrest, occurs within 5 minutes, and so is the assay.

### EXAMPLE 6

### Functional Binding Assay with Adherent Mouse ES Cells

By testing the adhesion of mES cells to various adhesion molecules and growth factors, it was found that the cytokine rhLIF (Chemicon, “LIF1010”) is capable of enhancing the binding of undifferentiated mES cells to an Ecadherin/FC chimera. In this example, slides were printed with replicate mixtures of Ecadherin and LIF, OSM, and IL6. The Ecadherin and 1×LIF (final) concentrations in the spotted mixture were 0.2 μg/μl and 0.002 μg/μl, respectively. Undifferentiated mES cells were dissociated thoroughly for 19′ using an enzyme-free dissociation solution (SpecialtyMedia) and re-suspended in 2 ml of incubation solution (DMEM/F12/PS+BIT+1×Selenium). 480 μl of cell suspension (at 5.8×10⁶ cells/ml) were applied to the printed region (˜324 mm²) on a “SuperAldehyde” slide and incubated for 15′ (the effect is usually observed within 10′). Following the incubation, the slide was washed in a slightly warmed DMEM chamber (˜30C).

The resulting pattern of attachment demonstrates a clear dose-dependent LIF-mediated enhancement of binding to Ecadherin (shown in FIG. 6). In this particular example, no binding was observed to Ecadherin alone, LIF alone, or Ecadherin-OSM and Ecadherin-IL6 spots. In some other cases, weak binding to Ecadherin (without LIF) could be observed but the binding was always enhanced by co-spotted LIF. The total protein concentration remains roughly the same for all the LIF dilutions, due to the negligible contribution of the LIF component to the overall spot mixture. The binding curve in FIG. 6 was constructed from two replicas on the same slide (regions #5 and 13). Error bars were estimated from spot to spot variations in the number of cells that bind to 10 identical spots.

Interestingly, exposure of the cells to 200 μg/ml (100×) of soluble LIF did not significantly increase the binding to spots containing low doses of printed LIF. Thus, the effect may require unusually high LIF concentrations (˜5 ng localized within about 5000 μm²), and/or immobilized LIF, and/or polarized exposure to LIF.

E-cadherin is known to be expressed on undifferentiated mES cells and is thought to be responsible for at least part of their clumping via homophilic interactions (between E-cadherins expressed on each of the cells). The cadherins form a major family of cell-cell adhesion, whose dynamics of expression correlates strongly with tissue segregation during development. Still, compared to integrins, very little is known about their activation. Therefore, the novel interaction between LIF and Ecadherin described here is of particular interest.

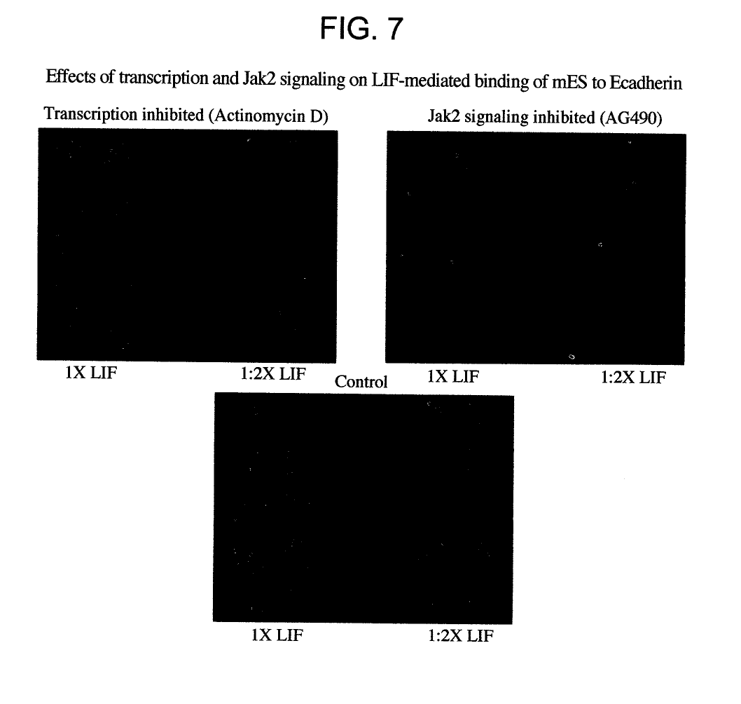

To further investigate the mechanism underlying this phenomenon, it was tested how the blocking of transcription and the inhibition of the canonical LIF signaling pathway affected the enhancement of ES cell binding to E-cadherin (shown in FIG. 7). Dissociated undifferentiated mES cells were treated for a short 20′ period with high doses (50 μg/ml (10×) and 1 mM (20×), respectively) of Actinomycin D (transcription inhibitor) and AG490 (blocks RTK by binding to its substrate binding sites thus inhibiting Jak-2 binding to the LIF receptor and gp130 cytoplasmic domains). (Both drugs are from CALBIOCHEM). Treated and untreated (control) cells were then applied to (separated) slides printed with replicas of Ecadherin-LIF dilutions. The resulting binding patterns show that transcription inhibition did not lead to a significant reduction in the number of bound cells (upper left versus the control below), but the blocking of the LIF pathway completely inhibited the binding (upper right). These results suggest that LIF signaling is responsible for Ecadherin activation by means of transcriptionally-independent pathway.

Taken together, the FBA results present important evidence that both the signaling and the capture molecules remain active on the slide following mechanical printing. Note that the two binding enhancers that were tested (SDF and LIF) signal through different mechanisms and different types of receptors. The FBA results demonstrate, in addition, the more general idea according to which one can use printed micro-environments to provide the cells with local, well-defined quantitative instructions that will induce nearby cell clusters to respond in a different manner. This principle may be applied to many other signal transduction pathways.

### EXAMPLE 7

### Long-Term, Spot-Directed Patterning of Mouse ES Cells

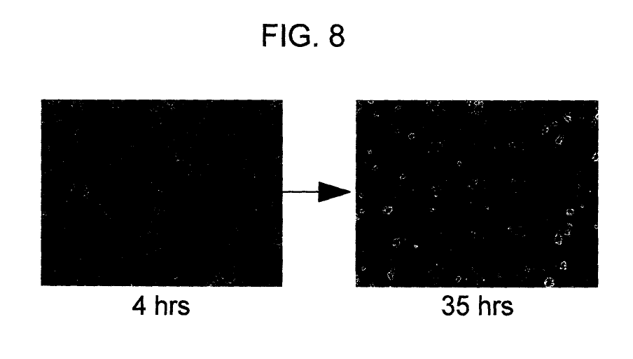

In cases where there is high non-specific binding to the slide, cells can still cluster on printed cues after time in culture. Shown in FIG. 8 are two time points (4 and 35 hrs following the initial incubation) of the same region containing 5 by 3 identical spots of 4:1 laminin and β-NGF (0.59 and 0.2 μg/μl, respectively) printed on a silylated slide from CEL Associate Inc., with inter-spot separation of 200 μm. Although this is also an aldehyde slide, the non-specific binding to this slide can be higher than to “SuperAldehyde” slides. In this particular example of mES on a silylated slide, the cells were initially randomly scattered all over the slide and ordered cell clusters could not be observed (left image). However, after 35 hrs of culturing in a GIBCO “NeuroBasal medium” supplemented with B27 serum-replacement, the cells became clustered on the laminin-based spots (right image). In addition, the remaining, background cells acquired a different morphology than those within the spots. Similar results were obtained with other culturing conditions and on different spot compositions.

For example, when the GIBCO Serum-replacement-based formulation (Knockout DMEM” with “Knockout Serum Replacement”, non essential amino acids, L-glutamine and PS) was used as the culturing medium, the cells still became clustered on the spots, but the background cells disappeared. The cells may migrate towards the spotted cues due to chemo-attractant properties of the latter or move around and arrest on the spots. In any case, the above example demonstrates an alternative scheme for self-patterning.

### EXAMPLE 8

### “On-Slide”, Medium-Ddependent Differentiation of mES Cells

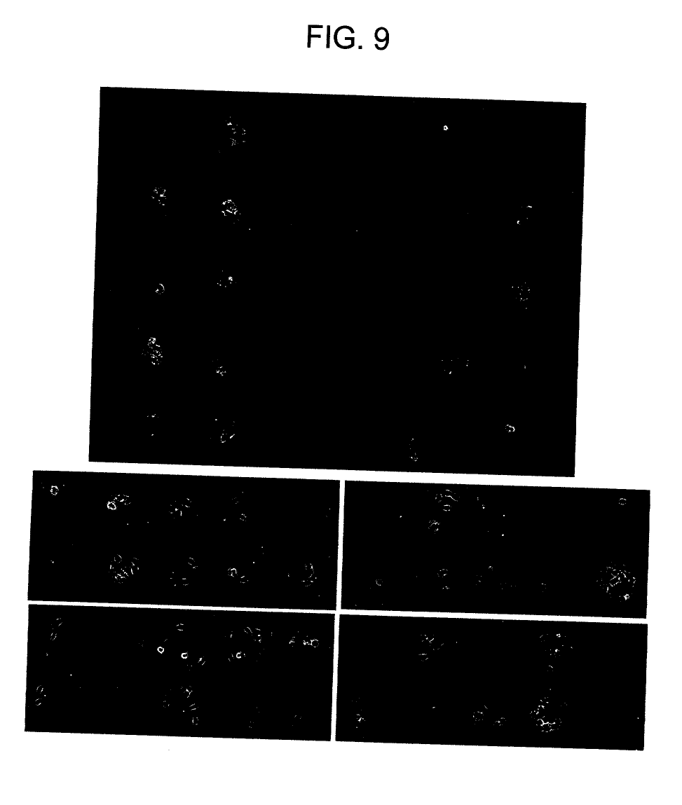

The example shown in FIG. 9 demonstrates the ability to induce the differentiation of mES cells in a relatively uniform, reproducible and medium-dependent manner. Initially undifferentiated R1 mES cells (18^{th} passage) were used. Prior to the experiment the cells were cultured and passaged according to standard methods. In short, the cells were cultured onto 1% gelatin coated dishes, fed every day with DMEM, 15% FBS, 1×(1000 U/ml) hLIF (Chemicon, LIF1010), 1×L-glutamine, 1×non-essential amino acids, 1×sodium pyruvate, 10 μM β-mercapto-ethanol, and 1×PS. The cells were split 1:5 every other day using trypsin-EDTA solution.

The cells were immobilized onto Ecadherin-LIF spots. The immobilization was performed as follows: cells were dissociated thoroughly for 17′ (Specialty Media Enzyme-Free Dissociation Solution). They were re-suspended in 2.4 ml (at 2.8×10⁶ cells/ml) of incubation solution (DMEM/F12/PS+10% BIT+1×, 0.67 μg/ml, Selenium), incubated (450 μl) on the slide for 12′, and washed in a big, slightly warmed, DMEM chamber.

The immobilized cells (on “SuperAldehyde” slides) were cultured for 42.5 hrs with (i) a defined medium for supporting mES cell survival on the slides (all four images above), which is a special formulation found to be capable of supporting viability and differentiation; and (ii) a serum-free-based, medium formulation from Gibco, supplemented with Retinoic acid (lower image). Defined medium was as follows: “Knockout DMEM” (from GIBCO), supplemented with 10% BSA-Insulin-Transferrin solution (“BIT9500” from Stem Cell Technologies), EGF and bFGF at 20 ng/ml each, 0.67 μg/ml (1×) Selenium, 1×L-glutamine, 1×non-essential amino acids solution, and 1×Pen/Strep (PS). Medium (ii) was as follows: “Knockout DMEM” with 15% “Knockout Serum Replacement”, 50 μM (100×) All-trans (ethanol dissolved) retinoic acid (RA) from SIGMA, 1×non essential amino acids, 1×L-glutamine and 1×PS.

Judging by the morphology, the resulting cell types differentiate in a medium-dependent manner. More importantly, for a given medium, the resulting cells look relatively uniform and lineage-restricted. In fact, the cells in the bottom image resemble epithelial cells and the ones in the upper images look dramatically different and resemble neural/glia progenitors. To obtain such differentiation on the slides, conditions had to be found that would allow cell survival while avoiding the formation of large cell clumps, within which the cells eventually differentiate spontaneously into cells of several different lineages (see FIG. 12 for a typical example).

The initial number of cells in each spot was reduced to about 10-15 (packing more cells would increase the uncontrolled cross talk between them, and less cells can compromise with their survivability and/or retention on the slide), and (ii) finding a substrate and spot compositions that would promote/favor differentiation, namely: a SuperAldehyde slide and 4:1 Ecadherin-LIF spots at final concentrations of 0.2 μg/μl and 0.002 μg/μl, respectively. The choice of defined minimal mediums capable of supporting cell survival and promoting differentiation (ideally without any bias towards a particular lineage) is also very important. The examples shown above (upper four images) demonstrate the usage of one such medium. It may be noted that DMEM/F12/BIT and neuroBasal-B27 based mediums, with or without common growth factors like EGF and FGF led to either cell necrosis or apoptosis).

### EXAMPLE 9

### “On-Slide”, Spot-Dependent Differentiation of mES Cells

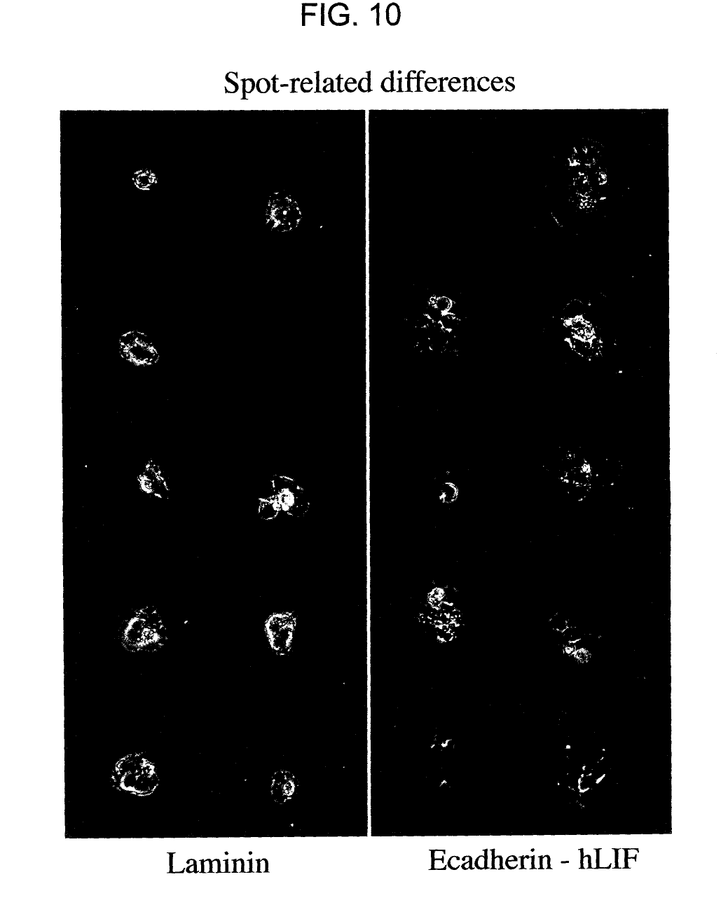

The data shown in FIG. 10 demonstrates the ability of a cell array to induce the differentiation of mES cells in a reproducible, spot-dependent manner. It shows that initially undifferentiated mES cells (as in FIG. 10), cultured on a single (SuperAldehyde) slide, but onto different spot compositions, can turn into morphologically distinct cell types. The experiment was performed as described in FIG. 10, with the Serum-Replacement-based medium.

Here, the cells captured on laminin spotted at 0.59 μg/μl (left image) formed growing, fairly undifferentiated, clumps, whereas those attached to Ecadherin-LIF (right image) are post-mitotic epithelial-resembling cells. By contrast, cell clusters on identical spots look uniform. This example demonstrates the feasibility of using local microenvironments for cell-fate manipulation. A straight forward approach would be to (i) immobilize dissociated undifferentiated mES cells on spots containing signaling cues (growth factors, cytokines, hormones) together with one or more ECM components (laminin, fibronectin, collagen, vitronectin, etc'), (ii) culture the cells on each slide with a different trial medium for up to several weeks, and (iii) screen for the desired phenotype by immuno-staining or any other standard readout. Note that tens to hundreds of identical slides can be printed in a single or consecutive print runs. Thus, the availability of many identical slides allows the exploration of different mediums in the “3^{rd} dimension”. Note also, that none of this is special to mES cells. An identical strategy could therefore be applied to hES cells or to differentiate hNSCs, for example, into dopaminergic neurons.

### EXAMPLE 10

### Uncontrolled Growth and Spontaneous Differentiation of mES Cells into Multiple Lineages

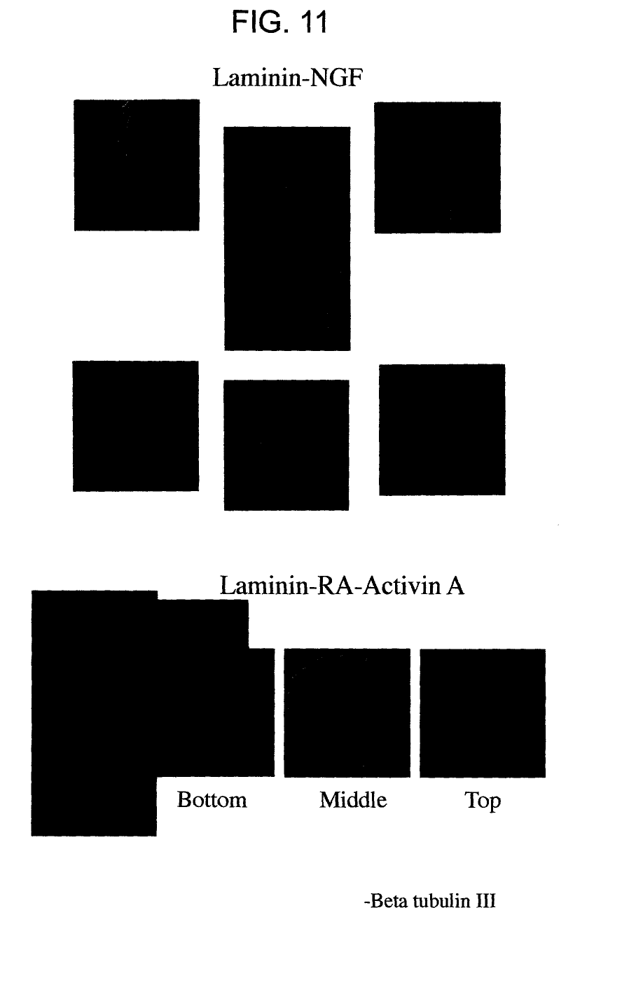

The two examples shown in FIG. 11 demonstrate the broad developmental potential of the same type of cells shown in FIGS. 9 and 10. R1 mES cells were immobilized on a Silylated slide as described in FIG. 10. The slide was then incubated for 1 day in a serum-replacement-based medium containing 0.5 μM all-trans RA and 0.5% DMSO following which the RA and DMSO were removed from the medium. Subsequently, the cells were fed every 2-3 days and fixed with 4% PFA after 16 days.

The major differences with respect to the previous examples (where the cells exhibited limited growth and restricted differentiation) were in the type of slide used (Silylated vs. SuperAldehyde”), and the density of cells that were initially applied to the slide (here 4.5×10⁶ cells on ˜324 mm² vs. 1.5×10⁶ on a similar area in FIG. 9). In addition, due to the high reactivity of the Silylated slide, as compared with the SuperAldehye, the actual density of cells that remained on the Silylated slide after the wash was much higher than on the corresponding example shown in FIG. 9.

Shown in FIG. 11 are several regions from a single slide stained for neuronal (β-tubulin class III), smooth muscle, and endothelial (Lectin) markers, and imaged by confocal microscopy. The structures that appear on the upper 6 images originated from initially undifferentiated cells that were captured on spots containing a 4:1 mixture of 0.59 μg/μl Laminin and 0.2 μg/μl β-NGF. The 3 images at the bottom reveal 3 different focal planes (bottom is the closest to the slide surface) of a chunk of tissue formed on spots containing a 4:1:1 mixture of 0.59 μg/μl Laminin, 50 μM (DMSO-dissolved) retinoic acid and 0.2 μg/μl Activin-A. The structures formed on the two different spot compositions are very different from one another, but both tissues were clearly stained positive for neurons, smooth muscles and endothelial cells, albeit with different distributions. Similarly, other combinations of growth factors and laminin (on the same slide) resulted in somewhat different cell distributions, most of them within a huge clump resembling the one shown in the 3 bottom images. By printing enough independent replicas of all the mixtures, one can determine the degree to which specific spot mixtures influence the resulting cell distributions.

### EXAMPLE 11

### Enhancement of Cell Proliferation by a Printed Cytokine

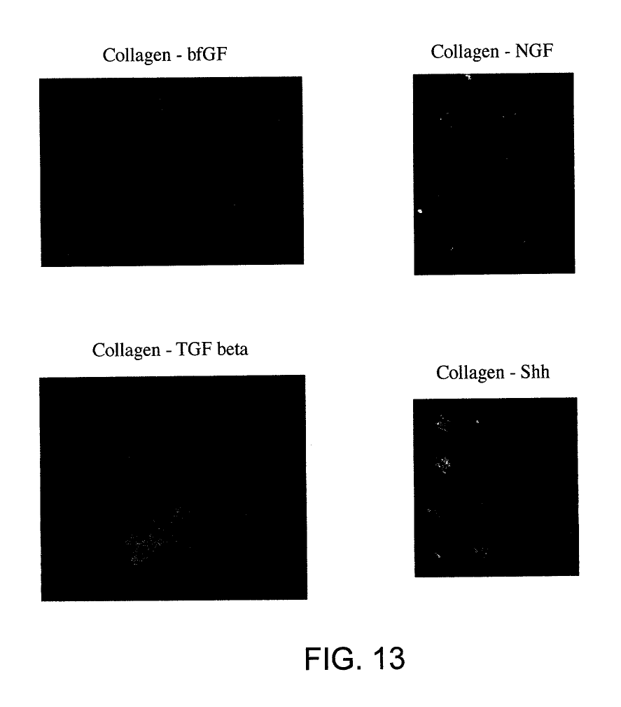

The following example shows that an immobilized cytokine/growth factor (printed together with a capture/glue molecule) can influence cell proliferation, as view by a standard BrdU incorporation assay. Shown in FIG. 12 are (human) Medullo-blastoma cell clusters attached to three ECM components (1 mg/ml collagen, 1 mg/ml Gelatin and 0.59 mg/ml Laminin) with or without rhLIF (right and left images images, respectively). The cells were grown in a serum-free medium (DMEM/F12/BIT/PSF+20 ng/ml EGF and 20 ng/ml PDGF), dissociated non-enzymatically for 10′, applied for 10′ to a SuperAldehyde slide (1.5×10⁶ cells onto a 650 mm²), printed with various mixtures of growth factors and ECM components, and washed with DMEM. After two days of culturing, 1×BrdU was added to the medium for 8 hours, following which the cells were fixed with 4% PFA and stained for BrdU (blue) and (β-tubulin type III—green). All three cases described in FIG. 13 show an increase in the number of BrdU incorporating cells on spots that contain LIF vs. those which did not.

### EXAMPLE 12

### Spot-Dependent Changes in Differentiation Marker Profile of Human Medullo-Blastoma

The interaction of cell with different micro-environments can lead to different cell-fate decisions. The latter can be viewed as a change in color profile when stained against various differentiation markers. Shown in FIG. 13 are four 10×fluorescent images of Medullo-blastoma cell clusters attached (on a SuperAldehyde slide) to collagen type IV (at 1 mg/ml), supplemented with different growth factors (bFGF, NGF, TGF-β, and Shh). The cells were cultured for two days in a defined medium (as described for FIG. 11), fixed with 4% PFA, and stained with antibodies against the following differentiation markers: glia (GFAP—in blue), neuronal (β-tubulin type III—green) and immature neural progenitors (Nestin—red). The scanned image indicates that each of the 4 spot mixtures has a distinct color profile. In three of these mixtures (namely, bFGF, NGF, and Shh), the clusters are of similar size, which makes it unlikely that the changes in color profile are due to differences in cluster size.

What is claimed is:

1. A method of profiling cells, the method comprising:
contacting a population of cells with a microarray, wherein said microarray comprises a pattern of spots of probes stably associated with the surface of a solid support, wherein the density of spots is at least 500/cm² and not more than 10,000/cm², and
determining the effect of said probes on said cells in a site specific analysis.

2. The method of claim 1 wherein the density of spots is at least 1000/cm².

3. The method according to claim 1, wherein said microarray comprises a plurality of different polypeptide probes.

4. The method according to claim 1, wherein said site specific analysis comprises determining a change in the phenotype of cells bound to said microarray.

5. The method according to claim 1, wherein said cell population comprises a single cell type.

6. The method according to claim 1, wherein said cell population comprises multiple cell types.

7. The method according to claim 5, wherein one or more of said cell types are differentially labeled with a detectable marker prior to said contacting step, and wherein said site specific analysis detects the presence of said marker.

8. The method according to claim 1, wherein said cell population comprises a single cell type.

9. The method according to claim 7, wherein said cells are labeled with a detectable marker, and wherein said site specific analysis detects the presence of said marker.

10. The method according to claim 1, wherein said microarray comprises polypeptides to which said cells will bind.

11. The method according to claim 7, wherein said microarray further comprises one or more spots comprising signaling probes.

12. The method according to claim 11, wherein said signaling probes comprise candidate pharmacologically active drugs.

13. The method according to claim 11, wherein said signaling probes comprise a peptide library.

14. The method according to claim 11, wherein said signaling probes are co-printed with binding probes.

15. The method according to claim 11, wherein said signaling probes are adjacent to binding probes.

16. The method according to claim 11, wherein said signaling probes comprises candidate agents

17. The method according to 11, wherein said signaling probes cause a change in the ability of said cells to bind to said microarray, and wherein said site specific analysis comprises determining the ability of cells to bind to a polypeptide in said array in the presence or in the absence of said signaling probe.

18. The method according to claim 4, wherein said cells are stem cells or progenitor cells, and wherein said change in the phenotype of cells bound to said microarray comprises detection of differentiation, de-differentiation or trans-differentiation of said cells.

19. The method according to claim 4, wherein said change of phenotype comprises detection of cell migration.

20. The method according to claim 4, wherein said change of phenotype comprises detection of secreted proteins.

21. The method according to claim 4, wherein said cell population comprises multiple cell types, and wherein said change in phenotype comprises detection of changes induce by interaction between two or more different cell types.

22. A method of profiling cells, the method comprising:
contacting a population of cells with a microarray, wherein said microarray comprises a pattern of spots of polypeptide probes stably associated with the surface of a solid support, wherein the density of spots is at least 500/cm² and not more than 10,000/cm², and wherein the polypeptides are other than antibodies or fragments thereof; and
determining binding of said cells to said polypeptide probes in a site specific analysis.

23. The method according to claim 22, wherein said microarray comprises a plurality of different polypeptide probes.

24. The method according to claim 22, wherein said cell population comprises multiple cell types.

25. The method according to claim 24, wherein one or more of said cell types are differentially labeled with a detectable marker prior to said contacting step, and wherein said site specific analysis detects the presence of said marker.

26. A method of profiling cells, the method comprising:
contacting a population of cells comprising multiple cell types differentially labeled with a detectable marker, with a microarray, wherein said microarray comprises a pattern of spots of polypeptide probes stably associated with the surface of a solid support, wherein the density of spots is at least 500/cm² and not more than 10,000/cm²; and
determining differential binding of said cells to said polypeptide probes in a site specific analysis.

27. A microarray comprising a pattern of spots of polypeptide probes other than antibodies or fragments thereof stably associated with the surface of a solid support, wherein the density of spots is at least 500/cm² and not more than 10,000/cm² and a population of cells bound to said polypeptide probes.

28. A microarray according to claim 27, wherein said solid support is a three-dimensional gel.

29. A microarray comprising a pattern of spots of polypeptide probes stably associated with the surface of a solid support, wherein the density of spots is at least 500/cm² and not more than 10,000/cm², and a population of cells comprising multiple cell types differentially labeled with a detectable marker, bound to said polypeptide probes.
